(19) United States
(12) Patent Application Publication
(10) Pub. No.: US 2011/0251594 A1
(43) Pub. Date: Oct. 13, 2011

(54) ARTICLE AND METHOD FOR OPENINGS IN LUMENS

(76) Inventor: Norman Godin, Geneva (CH)

(21) Appl. No.: 12/160,118

(22) PCT Filed: Jan. 13, 2006
(86) PCT No.: PCT/US06/01181

§ 371 Date: Jul. 7, 2008

(51) Int. Cl. A61M 25/00

(52) U.S. Cl. 604/528

(57) ABSTRACT

A system and method for measuring the cross sectional diameter of a body lumen are disclosed. A catheter tube having a proximal end, a distal end, and two or more strips formed from equal length parallel slits in the body, the strips normally parallel when the catheter tube is not stressed, the strips bowing outwardly from the tube when the distal end of the tube is stressed in a direction toward the proximal end of the catheter tube is inserted through an opening in a body, means such as a central tube or wire are pulled in a proximal direction to stress the catheter tube and thereby bow the strips outwardly in order to measure an opening such as a hiatal hernia.

### FIELD OF THE INVENTION

The present invention relates to the field of medicine, particularly to measuring openings, calibrating and fitting devices in patients.

### BACKGROUND OF THE INVENTION

We have described in U.S. Pat. No. 5,861,036 and U.S. Pat. No. 6,764,518 a new medical device to treat GERD (Gastro-esophageal Reflux Disease). This device consists of a ring placed in the lower esophagus or in a hiatus hernia of a patient with a tubular valve placed in the stomach at the cardia.

In order to keep the device in place in the lower esophagus or hiatus hernia, it is most important that the appropriate size of ring is used to place the device in the lower esophagus. If the ring is too large, it will not deploy or exert excessive pressure on the mucosa of the lower esophagus with the risk of creating ulcerations. If the ring is too small, it will fall in the stomach. As the ring will be attached to the wall or the esophagus or hernia with fasteners, tilt-tags, or other means, it is very important to have a right fit between the diameter of the lower esophagus or of a hiatus hernia and the diameter of the device.

The diameter of the lower esophagus or of a hiatus hernia could, in theory, be measured by imaging devices as a CT scan or an MRI. However, alternatives to using a CT scan or an MRI are desired in view of the costs involved and, in the case of CT, exposure to radiation. The lower esophagus and hiatus hernia are virtual cavities that are not completely expanded between meals or when they are not insufflated as done systematically during endoscopy, and therefore there is an advantage to endoscopy versus CT or MRI.

During endoscopy with a standard flexible gastroscope or video-gastroscope, insufflation is systematically performed through the air/water channel by the endoscopist. This insufflation allows passage of the gastroscope and careful examination of the walls of a hollow organ such as the lower esophagus or hiatus hernia.

Various body lumen measuring devices and methods have been proposed and used in the prior art, but none has been found acceptable for the special requirements related to measuring the lower esophagus so that a properly sized GARD can be placed. Most of the prior body lumen measuring devices and methods are difficult, expensive, and suffer from other problems even for their intended applications such as measuring the inside diameter of a blood vessel, enlarging a partially occluded artery lumen, measuring the interior lumen diameter in an esophagus, or measuring openings in bone tissue.

Korotko, et al., U.S. Pat. No. 6,450,976 disclosed an apparatus for measuring the inside diameter of a blood vessel having a long and flexible tube, measuring wires, and a knob comprising an inner barrel and outer barrel with a scale operative to show the relative position of the inner barrel with respect to the outer barrel.

Fogarty, U.S. Pat. No. 4,328,811, disclosed a catheter with an inflatable-deflatable balloon element to radially enlarge a partially occluded artery lumen, including a calibrator oval to internally gauge the enlarged lumen.

Colvin, et al., U.S. Pat. No. 5,010,892, disclosed a body lumen measuring instrument for insertion into a body passageway such as a bronchial tube in order to determine the interior lumen diameter or axial length of the passageway at a predetermined location, comprising a flexible sleeve having a handle secured to one end and a cable slidably positioned within the sleeve secured at one end to a slide actuator within the handle and at the other end terminates in a probe adapted for determining body passageway size under endoscopic observation. A measurement scale s associated with an indicator on the slide actuator in order to display the passageway size.

Hinnenkamp, U.S. Pats. 5,814,098 and 6,110,200, discloses an apparatus that can be adjusted through a range of diameters to measure an anatomical tissue annulus. The apparatus includes an elongated support member having a proximal end and a distal end. An operator actuated movable member is joined to the proximal end of the elongated support member while an adjustable member is joined to the distal end of the elongated support member. The adjustable member has a reference axis and an outer curved surface selectively positionable in response to the operator actuated member and the outer curved surface can be selectively positioned between an inner position proximate the reference axis and an outer position spaced apart from the reference axis.

Imran, U.S. Pat. No. 5,396,887, discloses an apparatus for performing an operation inside a body in the vicinity of a wall which includes a flexible elongate member having proximal and distal extremities, the distal extremity in the shape of a wire basket to be inserted into the body for performing the operation, having pressure sensors for sensing the force being applied to the distal extremity in bringing it into engagement with the wall so that the force being applied to the distal extremity can be controlled.

Shaffer, U.S. Pat. No. 5,702,401, discloses an intra-articular measuring device including a hollow handle defining a first passageway, and a hollow tube extending from the distal end of the hollow handle, the tube having a second passageway extending therethrough. The hollow tube carries a projection near its distal end for seating on a first selected region of a first bone, such as a tibial insertion point on a tibial plateau. The device further includes a probe slidably disposed within the first and second passageways, the hollow tube having a curvature sufficient to direct a distal end of the probe to a second selected region on a second bone, such as a femoral insertion point on a femur, to enable measurement of the distance between the first and second selected regions.

Kuzmak, et al., U.S. Pat. No. 4,686,288, disclosed a calibrating apparatus adapted to be inserted into and proceeded within the stomach comprising a single-lumen tubing which has a first opening extending therethrough which is located at a selected distance from the proximal portion and a second opening which extends therethrough and which is located a predetermined distance from the distal tip, an elongated, thin-walled sensor having a circular-cross section and a cavity within the interior thereof and which is operatively coupled in a sealing relationship to the distal tip wherein the thin-walled sensor is capable of having its cross-sectional diameter varied in response to a force applied to the exterior of the sensor to vary the pressure of a fluid within the cavity, a balloon surrounding the exterior of said single-lumen tubing at the preselected location which encloses the second opening and an elongated tubing member which extends through the single-lumen tubing, through the first opening and into communication with the second opening

Monfort, et al., U.S. Pat. No. 4,890,611, disclosed radially expansible, helically configured wire loops to grip and shear arteriosclerotic deposits from the lumen of an artery wall. The loops are pulled through the artery by means of a handle which is freely rotatable.

Mathews, et al., U.S. Pat. No. 6,427,351 disclosed a device for measuring distances between two points in a surgical setting which comprises an extension for extending into the body through an incision, the extension having a distal tip for insertion into the body and a proximal handle for remaining outside the body; a first measuring end and a second measuring end at the distal tip, the first and second measuring ends at a predetermined angle from each other and defining a distance between the first and second measuring ends, the first measuring end being movable relative to the distal tip, the first and second measuring ends being rounded; and an actuator at the proximal handle for moving the first measuring end relative to the distal tip.

Murphy, U.S. Pat. No. 5,902,308, discloses a device for determining cross-sectional dimensions of a blood vessel by first inflating a balloon catheter within the lumen until the balloon diameter matches the lumen diameter and a measurement element which indicates the expanded balloon cross-sectional area, circumference, or diameter with an optional fluoroscopic, radiographic, or ultrasound indication of the cross-sectional dimension or determined after deflation and removal of the catheter.

### SUMMARY OF THE INVENTION

It is an object of this invention to provide a simple system and method for measuring openings in hollow organs and other parts of human bodies.

Another object is to provide a device which can be used with an endoscope or gastroscope to measure the hiatus hernia, i.e., part of the top of the stomach sliding into the chest, so that an appropriate sized ring can be used to place or secure a tubular valve in the hiatus hernia or lower esophagus.

These objects, and others which will become apparent from the following disclosure and drawings, are achieved by the present invention which comprises in one aspect a catheter tube which is adapted to pass through the working channel of a endoscope or gastroscope that can be used under visual control to measure the diameter of a hollow organ such as the esophagus, hiatus hernia, duodenum, small bowel or colon and rectum. The catheter tube can be plastic or metal.

The opening in conventional adult gastroscopes is usually 2.8 mm, but can vary between 2.0 mm and 5.0 mm for non-conventional gastroscopes such as pediatric gastroscopes or therapeutic endoscopes with larger channels. A video gastroscope can be used to assist in visualizing the measurement process with devices of the invention.

The system of the invention can be used to measure diameters in other tubular organs such as the larynx, trachea, bronchi, urinary tract such as urethra or ureters, or a blood vessel.

The device comprises a catheter tube and a handle. The catheter tube is placed through the working channel of the gastroscope until the last few inches or centimeters are visible. The lower esophagus or hiatus hernia are insufflated and the calibration basket is opened by pulling on the handle. The calibration basket is opened until the loops touch the mucosa of the hernia or wall of the organ measured on each side. The diameter of the opening is then read on the handle or the handle is opened up to a graduation that is read.

Once the device is pulled out of the endoscope, the basket is opened to the same graduation mark and the diameter is read on a calibration sheet.

One advantage of this system is the easy use through a conventional working channel of a gastroscope and the possibility to measure relatively large diameters, up to 30 mm with a device measuring 2 mm in diameter. This is not possible with a balloon catheter, even made in the most inflatable material such as silicone, where the largest increase is about 1000%, that is for a 2 mm. diameter, a silicone balloon can at most be dilated up to 20 mm. The typical range of the rings used to hold the tubular anti-reflux device called the GARD is 26 mm to 30 mm. Furthermore, balloons used for endoscopy are quite expensive.

Therefore the calibration basket can be used as a moderately priced, accurate solution to measure large and small diameters through an endoscope. The calibration basket is not limited to measuring the gastro-intestinal (GI) tract but can be used to place prothesis in the larynx, trachea, bronchi, or the urinary tract, and can be used to measure other body lumens for purposes other than placing a prothesis

The calibration sheet is simply holes that are punched out in a sheet of carton or plastic with the different diameters we use. Any appropriate selection of hole sizes can be used, depending on the normal range of the lumen being measured. For example, in some embodiments useful for measuring the GI tract, the holes can be 22 mm to 30 mm at 2 mm intervals, whereas in other embodiments measurements ranging from 2 mm to 20 mm at 2 mm intervals can be used.

### BRIEF DESCRIPTION OF THE DRAWINGS

FIG. 1 is a prospective view of a section of a body lumen measuring device according to the invention in a stressed condition with strips bent outwardly.

FIG. 2 is a cross-sectional view through 2-2 of FIG. 1 showing in internal tube within a catheter tube body.

FIG. 3 is a prospective view of the handle portion and the distal portion of a body lumen measuring device according to the invention, with the central portion not shown.

FIG. 4 is a prospective view of the indicator portion 4 of FIG. 3.

FIG. 5 is a front elevational view of a calibration sheet used in certain embodiments of the invention.

### DETAILED DESCRIPTION

The present invention provides methods and apparatus for determining cross-sectional dimensions such as the internal diameter of a body lumen. The methods and apparatus are especially useful for sizing intraluminal prosthesis such as gastric anti-reflux devices, and can be used to measure a variety of lumens, including, for example, lower esophagus, hiatus hernia, cardia, larynx, bronchus, ureter, urethra, and blood vessels.

Although the invention is capable of many alternative embodiments, one such embodiment is illustrated in the drawings.

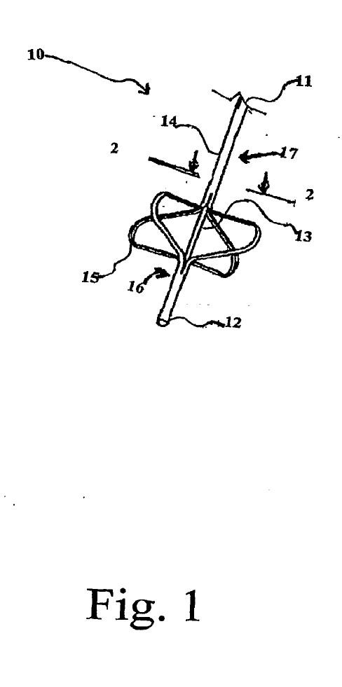

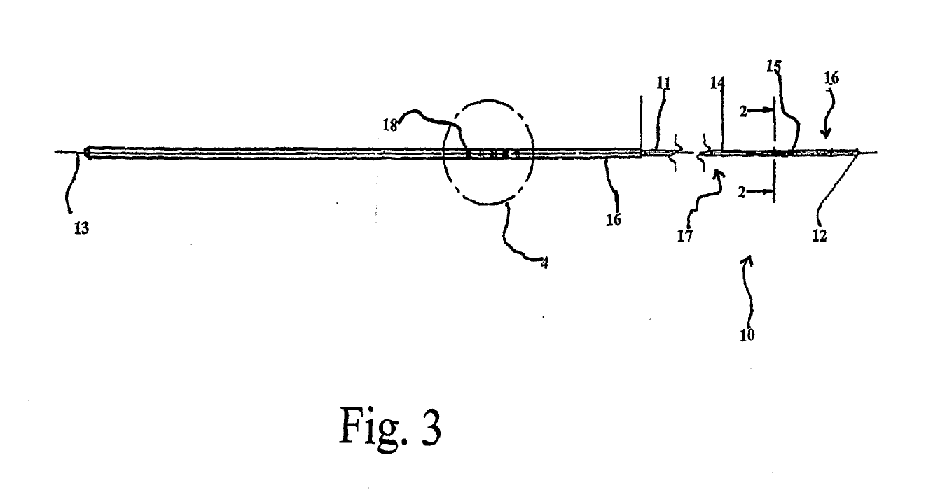

Referring first to FIG. 1, a section 10 of a measuring device is shown wherein a section of plastic catheter tube 14 has a distal end 12, a distal portion 16, a proximal end 11 and proximal portion 17. A central tube 13 is welded or otherwise bonded at distal portion 16 of plastic catheter tube 14; and slides within proximal portion 17. The central tube 13 is welded or otherwise bonded within clear plastic handle 16 (FIG. 3) which has a lumen within which plastic catheter tube 14 is slidably engaged. Thus when handle 16 is pulled in the proximal direction while holding plastic catheter tube 14 still, the strips 15 are stressed and bow outwardly to a degree corresponding to the linear distance which the central tube 13 is moved in the proximal direction in relation to the plastic catheter tube 14, and are bowed inwardly when the central tube is moved in the distal direction in relation to the plastic catheter tube 14. The amount of bowing and resultant expansion of diameter depends on the amount the central tube 14 is moved. The illustrated embodiment has four strips 15, created by four parallel and equal length slits along the length of the plastic catheter tube 14, thus the strips 15 are cut from the plastic catheter tube 14 and have, or course, the same plastic composition as the plastic catheter tube 14. The number of strips 15 can be as few as two and as many as 8, and are not necessarily formed from the plastic catheter tube by slitting the plastic catheter tube 14. Furthermore, the member referred to as plastic catheter tube 14 can be made of other materials, for example metal.

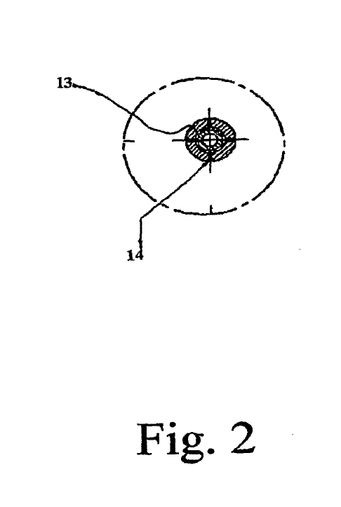

FIG. 2 is a cross-sectional view through 2-2 of FIG. 1, showing the central tube 13 within plastic catheter tube 14. In the cross-section through 2-2, the central tube 13 is slidably engaged within plastic catheter tube 14, but in the distal portion 16 of FIG. 1, the central tube 13 is welded, crimped, glued, or otherwise bonded to plastic catheter tube 14.

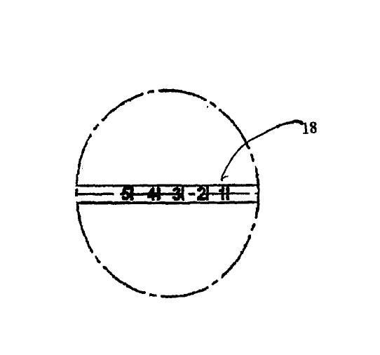

FIG. 3 illustrates a clear plastic handle 16 which is bonded to the proximal end of central tube 14, with the strips 15 illustrated in this view in unstressed, unbowed position rather than stressed and outwardly bowed as illustrated in FIG. 1. Indicator markings 18 in area 4 of handle 16, illustrated in magnified view in FIG. 4, are used to measure the distance central tube 13 has moved in the proximal direction relation to plastic catheter tube 14, which in turn causes the strips 15 to bow outwardly as illustrated in FIG. 1. The indicator markings 18 are illustrated with hashes where are numbered 1 through 5, but letters or other markings can be used.

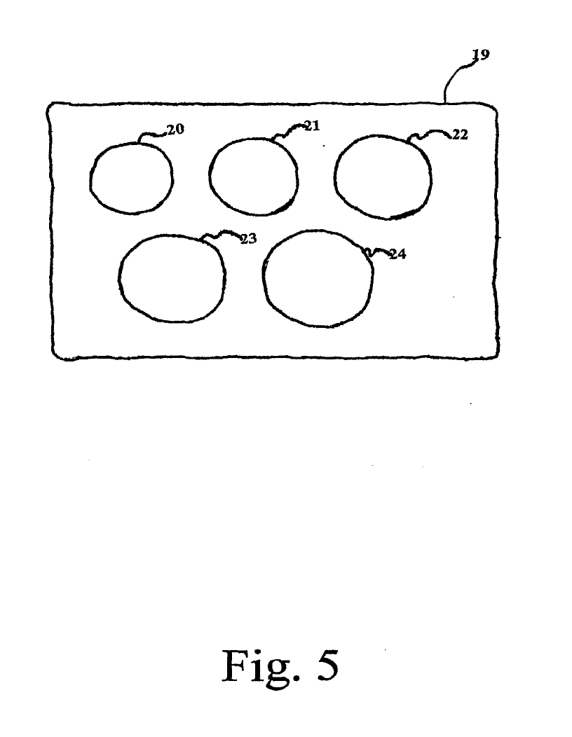

FIG. 5 illustrates a calibration sheet 19 having holes 20, 21, 22, 23, and 24, which have diameters of 22, 24, 26, 28, and 30 mm respectively. This calibration sheet 19 can be used, for example, to determine the cross-sectional diameter of a body lumen by bowing the strip section 15 of the measuring device, after it has been withdrawn from the gastroscope or endoscope, to the same diameter it was bowed when in the lumen being measured. The indicators 18 are used to indicate the extent the central tube 13 must be moved in a proximal direction after the device has been removed from the gastroscope or endoscope in order to replicate the measurement taken within the body lumen. If the central tube 13 was moved to indicator number 5, for example, the strips 15 would have bowed to a 30 mm diameter, for example, and so when the strips 15 are unstressed and the device is removed from the endoscope or gastroscope, the strips 15 can be restressed the same amount by returning the central tube marker to the same indicator number 18, and then the diameter of the bowed strips 15 can be calibrated using the calibration sheet 19.

To illustrate one embodiment of operation of the device, the measuring device can be placed in a conventional gastroscope or endoscope to place the strip portion 15 within a lumen to be measured, for example a hiatus hernia, in order to measure the cross-sectional diameter and thereby properly size a GARD as illustrated in my U.S. Pat. No. 6,764,518 for insertion in a second procedure. The strips 15 are in their unstressed position when they are first placed at the lumen to be measured, and then the central tube 13 is manually moved in a proximal direction until the strips 15 bow outwardly to the diameter of the lumen, as can be seen through the gastroscope or endoscope. The amount the central tube 13 has moved is determined by observation of the indicators 18, for example the central tube marker may move to indicator marking 3 as shown in FIG. 4.

While the invention has been described and exemplified in detail herein, various modifications, alternatives, and improvements should become apparent to those skilled in the art without departing from the spirit and scope of the invention.

What is claimed is:

1. A body lumen cross sectional dimension measuring device comprising:
a. a catheter tube having a proximal end, a distal end, and two or more strips formed from equal length parallel slits in the body, the strips normally parallel when the catheter tube is not stressed, the strips bowing outwardly from the tube when the distal end of the tube is stressed in a direction toward the proximal end of the catheter tube,
b. means for stressing the catheter tube; and
c. means for calibrating the bowing outwardly.

2. The device of claim 1 wherein the means for stressing the catheter tube to cause the strips to bow outwardly comprises a central wire or tube having a proximal end and a distal end, the central wire or tube disposed within the lumen of the catheter tube body, the distal end of the wire or tube being attached to the distal end of the catheter tube, the proximal end having means for moving the central wire or tube in a proximal direction in relation to the catheter tube.

3. The device of claim 3 wherein the means for moving the central wire or tube in a proximal direction in relation to the catheter tube comprises a handle on or near the proximal end of the central wire or tube.

4. The device of claim 2 wherein the means for calibrating the bowing outwardly comprises markings on the catheter tube which indicate the degree to which the central wire or tube has moved in a proximal direction in relation to the catheter tube.

5. The device of claim 2 wherein the means for calibrating the bowing outwardly comprises means to correlate the cross sectional diameter formed by the outwardly bowed strips to the means for stressing the catheter tube.

6. The device of claim 2 comprising means to determine the extent to which the strips are bowed comprising graduation markings on the catheter tube and an indicator marking on the central wire or tube.

7. The device of claim 2 comprising means to determine the extent to which the strips are bowed comprising graduation markings on the catheter tube and an indicator marking on the central wire or tube and a calibration sheet having a plurality of holes of varying sizes.

8. The device of claim 1 wherein the strips return to their normal position when the central wire or tube is moved in a distal direction.

9. The device of claim 1 wherein the catheter tube has a length sufficient to extend from a distal location outside the body to a proximal location where cross-sectional measurement is desired.

10. The device of claim 1 wherein the catheter tube comprises two to five slits which form two to five lengthwise sections.

11. The device of claim 1 having four slits which form four strips.

12. The device of claim 1 wherein the means for stressing the catheter tube to cause the strips to bow outwardly comprises a central wire or tube having a proximal end and a distal end disposed within the catheter tube, the distal end of the wire or tube being attached to the distal end of the catheter tube, the proximal end having means for moving the central wire or tube in a proximal direction in relation to the catheter tube; and wherein the catheter tube has a length of about 500 to 2000 mm, an outer diameter of about 1 to 3 mm, an inner diameter of about 0.5 to 2 mm, the lengthwise sections have a length of about 20 to 50 mm, the lengthwise sections disposed about 1 to 10 mm from the distal end where the central wire or tube is attached to the catheter tube, and an outer diameter of the central wire or tube which is slightly less than the inner diameter of the catheter tube.

13. The device of claim 1 adapted for measuring the cross sectional diameter of a body lumen selected from the group consisting of lower esophagus, hiatus hernia, cardia, larynx, bronchi, urinary tract, and blood vessel.

14. The device of claim 1 wherein the catheter tube is plastic.

15. The device of claim 1 wherein the catheter tube is metal.

16. A method of measuring the diameter of a body lumen comprising providing a device according to claim 1, inserting the catheter tube through an opening in the body so that the strips are located at the body lumen where a cross sectional dimension is to be measured, bowing the strips outwardly by stressing the catheter tube in a proximal direction, calibrating the degree to which the catheter tube has been stressed to the resulting cross sectional diameter formed by the outwardly bowed strips, unstressing the catheter tube in a distal direction so as to unbow the strips inwardly, removing the device from the opening in the body.

17. The method of claim 16 wherein the means for stressing the catheter tube to cause the strips to bow outwardly comprises a central wire or tube having a proximal end and a distal end, the central wire or tube disposed within the lumen of the catheter tube body, the distal end of the wire or tube being attached to the distal end of the catheter tube, the proximal end having means for moving the central wire or tube in a proximal direction in relation to the catheter tube, comprising moving the central tube in a proximal direction in relation to the catheter tube to stress the strips and bow the strips to a cross sectional diameter matching the cross sectional diameter of the body lumen.

18. The method of claim 17 comprising providing means on the catheter tube and the central wire or tube to determine the amount the central tube has been moved in a proximal direction in relation to the catheter tube, correlating the cross sectional diameter of the bowing of the strips to the amount the central tube is moved longitudinally in a proximal direction in relation to the catheter tube.

19. The method of claim 16 wherein the body lumen is selected from the group consisting of lower esophagus, hiatus hernia, cardia, larynx, bronchus, ureter, urethra, and blood vessel.
